(19) United States
(12) Patent Application Publication
(10) Pub. No.: US 2010/0047121 A1
(43) Pub. Date: Feb. 25, 2010

(54) PROCESS AND APPARATUS FOR STERILIZING LIQUIDS FOR MEDICAL USE

(75) Inventors: Marco Fabiano, Calestano (Parma) (IT); Francesco Di Salvo, Calestano (Parma) (IT)

Correspondence Address: YOUNG & THOMPSON, 209 Madison Street, Suite 500, Alexandria, VA, 22314, US

(73) Assignee: G.E.A.F. S.R.L., Calestano (IT)

(21) Appl. No.: 12/530,372

(22) PCT Filed: Feb. 29, 2008
(86) PCT No.: PCT/EP08/52489

§ 371 Date: Sep. 8, 2009

(30) Foreign Application Priority Data
Mar. 5, 2007 (IT) PR2007A000013

(51) Int. Cl. A61L 2/00; A61L 2/08; A61L 2/04

(52) U.S. Cl. 422/22; 250/453.11

(57) ABSTRACT

The novel sterilization process disclosed herein still relies on the sterilization action resulting from exposure of the liquids and inner surfaces of the medical article to appropriate temperatures for well-determined times, but the sterilization temperature shall be attained through generation of heat in the article itself and not through heat transfer from outside to inside. This shall be obtained through the generation of a medium to high frequency electromagnetic field, whose characteristics are precisely determined to reach the desired temperature in as little time as possible and to maintain it for the time required for safe sterilization of the article.

The present invention relates to a process and an apparatus for sterilizing liquids for medical use, particularly liquids held in rigid containers, e.g. made of glass, or flexible containers, such as pouches, or tubular medical articles made of polymeric plastic materials.

In prior art, medical liquids in rigid or flexible containers are sterilized by heat, i.e. by increasing liquid temperature to values of about 120° C. and maintaining such temperature for a time determined as a function of certain coefficients associated with biological parameters.

The heating process is carried out in autoclaves using steam or overheated water which comes in contact with the outer surfaces of the containers of infusion solutions or other medical liquids and heat such solutions to the desired temperature and for the time imposed by the various pharmacopeiae.

Such heating takes place by conduction or convection from outside to inside the container and suffers from a number of drawbacks, such as: uneven temperature distribution in the liquid mass, long-time maintenance of high temperatures in the products, which involves degradation of both container and liquid properties, mechanical stresses on containers, especially non-rigid containers, caused by internal pressure, possibly leading to failure of the container if such internal pressure is not adequately balanced with an equal external pressure.

In view of obviating such drawbacks, prior art sterilization systems that use steam or overheated water are inevitably complex, bulky and expensive.

Such systems have to be designed with a steam or overheated water generation system as a heat source, require pressure regulation systems in the autoclaves for balancing internal pressures in the containers and involve the use of large hermetic receptacles with adequate wall thicknesses to withstand the mechanical stresses caused by such pressures.

Thus, as mentioned above, these systems are very large, complex and costly and involve difficult handling and operation.

On the other hand, tubular medical articles are sterilized by exposure to toxic gases, typically ethylene oxide, in special autoclaves or by exposure to radiation from radioactive materials.

Ethylene oxide sterilization uses a toxic gas which is introduced in an article-containing autoclave. Ethylene oxide penetrates the articles and destroys bacterial growth therein. In addition to the precautions required for safe and hazardless use of this gas, removal of the slightest trace of gas, especially the highly toxic ethylene glycol, from the article must be ensured. This requires a long and burdensome product ventilation step.

Radiation sterilization consists in exposing the articles to radiation from special radioactive facilities. A very small number of such facilities is currently available, wherefore expensive and time-consuming material handling is required to carry the materials to sterilization radioactive facilities and back. Furthermore, in addition to destroying bacterial growth, radiation significantly degrades the article, thereby strongly affecting the initial product quality.

In short, prior art sterilization systems are burdensome, expensive, involve many risks and dangers and have a detrimental effect on the properties of the materials.

The above objects and advantages are fulfilled by the process and apparatus for sterilizing liquids for medical use, particularly liquids held in rigid containers, e.g. made of glass, or flexible containers, such as pouches, or tubular medical articles made of polymeric plastic materials according to the present invention, which is characterized as set out in the accompanying claims.

This and other features will be more apparent from the following description of a few embodiments, which are shown by way of example and without limitation in the accompanying drawings, in which:

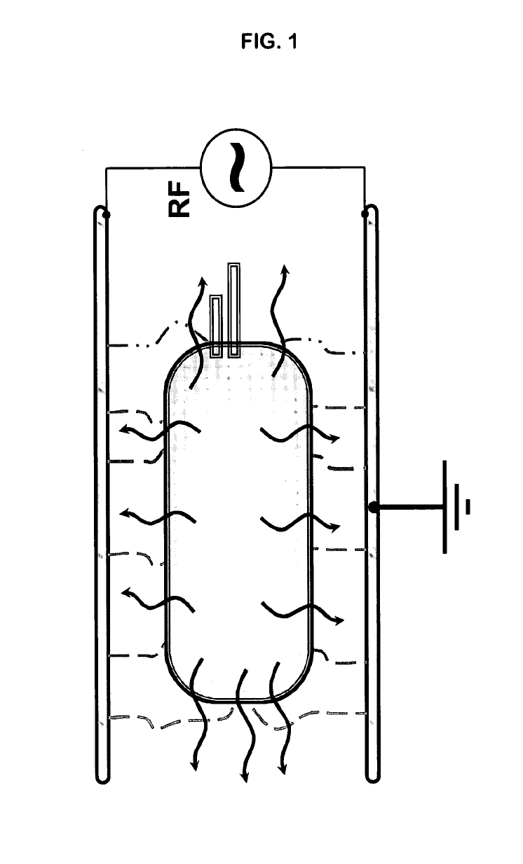

FIG. 1 shows an operation diagram of the present process in which the sterilization temperature is reached to generate heat in the article,

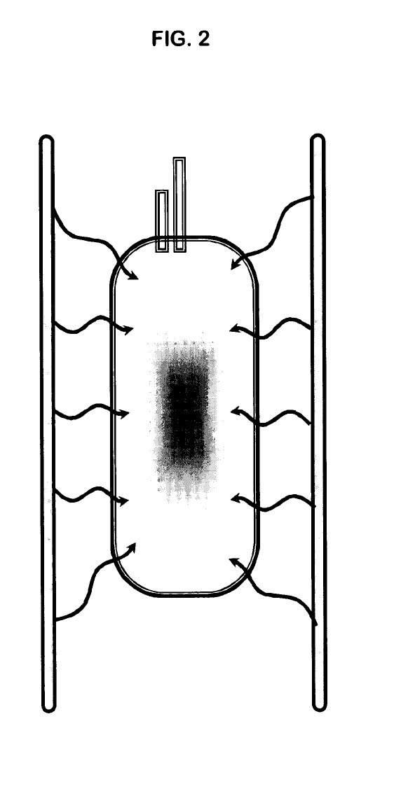

FIG. 2 shows an operation diagram of a classical process with heat generation from the outside.

The present invention relates to a process for sterilizing liquids for medical use, held in rigid (glass) containers or flexible containers (pouches or tubular medical articles made of polymeric plastic materials), which uses heat generating processes utilizing electromagnetic waves with frequencies in a range from 10 to 200 MHz. Such system will provide:
sterility of the liquids contained in bottles or pouches, required for various medical treatments, such as infusion, nutritional, dialysis treatments
sterility of tubular medical articles used in a large number of treatments, such as transfusion, hemodialysis, blood collection and separation, etc.

The novel sterilization process disclosed herein still relies on the sterilization action resulting from exposure of the liquids and inner surfaces of the medical article to appropriate temperatures for well-determined times, but the sterilization temperature shall be attained through generation of heat in the article itself (as shown in FIG. 1) and not through heat transfer from outside to inside (as shown in FIG. 2).

This shall be obtained through the generation of a medium to high frequency electromagnetic field, whose characteristics are precisely determined to reach the desired temperature in as little time as possible and to maintain it for the time required for safe sterilization of the article.

The main advantages achieved through such novel system include a simpler and less expensive sterilization process, lower thermal stresses and degradation of the sterilized product.

Another critical advantage consists in the destruction of any bacterial growth in the liquid or the surfaces of the sterilized articles, owing to the concentration of the electromagnetic field in the locations where organic materials are likely to be present.

Indeed, a stronger sterilizing effect is achieved, under the same average liquid temperature conditions, as compared with conventional prior art systems.

Reference to a totally different physical principle from that used in prior art leads to thorough redesign and reconversion of current technological process systems for sterilizing liquids for medical use and medical articles, thanks to the possibility of overcoming the three prior art sterilization processes (steam and overheated water, ethylene oxide, gamma or beta radiation) to use a single sterilization process using medium to high frequency electromagnetic fields.

In other words, the introduction of a novel sterilization system in this market involves the creation of new technological processes with great simplification and considerable savings.

The basic physical parameters, such as the frequency ranges and sequences, temperatures and times as mentioned above, associated with biological parameters describing the resistance of biological life forms and more heat-resistant spores show that the present novel sterilization system provides a reduction of both times and temperatures from 5 to 10% of the values as used in normal prior art heat sterilization processes, thereby involving a considerably reduced thermal degradation of the product.

These objects and advantages are fulfilled by the liquid sterilization process of the present invention, which is characterized by the annexed claims.

With the process of the present invention, the novel apparatus and facilities will be characterized as follows:
The products to be sterilized shall contain liquids with such a reference molarity as to have a minimum electrical conductivity above 150 microSiemens per cm,
The products to be sterilized shall be preferably individually packaged under vacuum in bags made of materials able to withstand temperatures above 100° C.,
The products to be sterilized shall be preferably placed in special non-metal containers before introducing them in the sterilization apparatus or facility,
An exemplary embodiment of an apparatus or facility consists of a parallelepipedal or cylindrical chamber in which the products can be placed on suitable non-metal carriages,
An exemplary embodiment of the apparatus or facility consists of a tunnel with a carpet conveyor,
Both chambers and tunnels shall be equipped with systems for generating electromagnetic fields having frequencies in a range from 10 to 200 MHz,
The frequency operation time can be either used by a single frequency or divided into cycles having different frequency values depending on the characteristics of the product to be sterilized,
The system for monitoring and controlling the generators of electromagnetic fields in the chambers or tunnels shall allow execution of programmed cycles of transmission of electromagnetic waves having various frequencies depending on quick and safe product sterilization requirements.

1. A process for sterilizing liquids for medical use, held in rigid, containers, e.g. made of glass, or flexible containers, such as pouches or tubular medical articles made of polymeric plastic materials, characterized in that it involves exposure of the liquids and inner surfaces of the medical article to heat, said sterilization temperature being attained through generation of heat in the article itself and not through heat transfer from outside to inside; said heat generating process utilizing electromagnetic waves with frequencies in a range from 10 to 200 MHz.

2. A process as claimed in claim 1, characterized in that it includes the step of placing the products to be sterilized in suitable non-metal containers before introduction thereof in the sterilization apparatus or facility.

3. A process as claimed in claim 1, characterized in that it operates on liquids having such a reference molarity as to exhibit a minimum electrical conductivity above 150 microSiemens per cm, said liquids being held in rigid or flexible containers.

4. A process as claimed in claim 1, characterized in that it operates on products individually packaged under vacuum in bags made of materials able to withstand temperatures above 100° C.

5. A process as claimed in claim 1, characterized in that the frequency operation time can be either used by a single frequency or divided into cycles having different frequency values depending on the characteristics of the product to be sterilized.

6. An apparatus or facility for carrying out the process as claimed in claim 1, characterized in that it comprises a parallelepipedal or cylindrical chamber in which the liquids to be sterilized, held in rigid or flexible containers, can be placed on suitable non-metal carriages.

7. An apparatus or facility for carrying out the process as claimed in claim 1, characterized in that it comprises a tunnel in which the liquids to be sterilized, held in rigid or flexible containers are conveyed on a carpet conveyor.

8. An apparatus as claimed in claim 6, characterized in that it comprises at least one system for generating electromagnetic fields with frequencies in a range from 10 to 200 MHz.

9. An apparatus as claimed in claim 8, characterized in that the system for monitoring and controlling the generators of electromagnetic fields in the chambers or tunnels shall allow execution of programmed cycles of transmission of electromagnetic waves having various frequencies depending on quick and safe product sterilization requirements.

10. An apparatus or facility for carrying out the process as claimed in claim 2, characterized in that it comprises a parallelepipedal or cylindrical chamber in which the liquids to be sterilized, held in rigid or flexible containers, can be placed on suitable non-metal carriages.

11. An apparatus or facility for carrying out the process as claimed in claim 3, characterized in that it comprises a parallelepipedal or cylindrical chamber in which the liquids to be sterilized, held in rigid or flexible containers, can be placed on suitable non-metal carriages.

12. An apparatus or facility for carrying out the process as claimed in claim 4, characterized in that it comprises a parallelepipedal or cylindrical chamber in which the liquids to be sterilized, held in rigid or flexible containers, can be placed on suitable non-metal carriages.

13. An apparatus or facility for carrying out the process as claimed in claim 5, characterized in that it comprises a parallelepipedal or cylindrical chamber in which the liquids to be sterilized, held in rigid or flexible containers, can be placed on suitable non-metal carriages.

14. An apparatus as claimed in claim 7, characterized in that it comprises at least one system for generating electromagnetic fields with frequencies in a range from 10 to 200 MHz.
